(19) United States
(12) Patent Application Publication
(10) Pub. No.: US 2024/0188847 A1
(43) Pub. Date: Jun. 13, 2024

(54) A SAMPLE COLLECTOR

(71) Applicant: IMEC VZW, Leuven (BE)

(72) Inventors: Bert VERBRUGGEN, Oud Heverlee (BE); Filip PAEPS, Leefdaal (BE); Boris UYTTERHAEGEN, Leuven (BE); Tobe WAUTERS, Leuven (BE)

(21) Appl. No.: 18/287,501

(22) PCT Filed: Apr. 14, 2022
(86) PCT No.: PCT/EP2022/060045

§ 371 Date: Oct. 19, 2023

(30) Foreign Application Priority Data
Apr. 19, 2021 (EP) 21169266.0

(51) Int. Cl. A61B 5/097

(52) U.S. Cl. CPC A61B 5/097

(57) ABSTRACT

The disclosure relates to a sample collector for collecting airborne particles exhaled by a human being, comprising: a sampling compartment for receiving a flow of air; a particle capturing substrate for capturing the airborne particles in the flow; an analysis compartment; and a substrate clamping device comprising an optical window and a support member; wherein the sample collector is reconfigurable between a sampling configuration in the sampling compartment for passage of the flow through the particle capturing substrate and an analysis configuration, wherein in the particle capturing substrate is arranged in the analysis compartment, the optical window is aligned with an aperture for allowing optical access to the particle capturing substrate is allowed; and
wherein, during reconfiguration from the sampling configuration to the analysis configuration, the substrate clamping device and the particle capturing substrate are moved together to the analysis compartment while the substrate clamping device is brought to the closed state.

### TECHNICAL FIELD

The present disclosure relates to a sample collector for collection of airborne particles. In particular, the present disclosure relates to collection of airborne particles, exhaled by a human being, for facilitating optical analysis of the airborne particles.

### BACKGROUND

Efficient collection of airborne particles exhaled by a human to facilitate analysis is of high interest in various applications. For instance, enabling detection of substances in a human breath may provide the possibility of screening for infectious diseases, such as influenza and severe acute respiratory syndrome coronavirus 2 (SARS-COV-2). Thus, collection of exhaled airborne particles and in an efficient and inexpensive way may allow screening to be performed frequently, allow quick identification of persons carrying disease, and consequently reducing spreading of the disease. Several diseases, such as influenza and SARS-COV-2, spread among humans through droplets and aerosols produced during breathing, blowing, talking, coughing, and sneezing. Thus, it would be of interest to provide a sample collector allowing efficient capture of airborne particles exhaled by a person and facilitating analysis of substances within the captured particles so as to identify whether the person carries a disease or not.

### SUMMARY

In light of the above, it is an objective of the present disclosure to provide a sample collector allowing simple and efficient collection of airborne particles exhaled by a human, facilitating analysis of the collected particles. A further objective is to facilitate a safe handling of the sample collector, e.g. to reduce a risk of contamination of the sample and/or spreading of collected airborne particles.

These and other objectives of the present inventive concept are at least partly met by the invention as defined in the independent claims. Embodiments are set out in the dependent claims.

In accordance with an aspect there is provided a sample collector for collection of airborne particles exhaled by a human being, comprising: a sampling compartment comprising an inlet for receiving a flow of air (e.g. from exhalation by the human being); a particle capturing substrate configured to capture the airborne particles in the flow of air; an analysis compartment wherein an aperture is defined in a wall of the analysis compartment; and a substrate clamping device comprising an optical window and a support member; wherein the sample collector is reconfigurable between a sampling configuration and an analysis configuration, wherein:
in the sampling configuration the particle capturing substrate is arranged in the sampling compartment such that passage of the flow of air through the particle capturing substrate is allowed; and
in the analysis configuration the substrate clamping device and the particle capturing substrate are arranged in the analysis compartment, the optical window is aligned with the aperture such that optical access to the particle capturing substrate is allowed, and the substrate clamping device is in a closed state such that the particle capturing substrate is clamped between the optical window and the support member; and
wherein, during reconfiguration from the sampling configuration to the analysis configuration, the substrate clamping device and the particle capturing substrate are configured to be moved together to the analysis compartment while the substrate clamping device is brought to the closed state.

The sample collector allows a human being to breath or blow into the sample collector such that particles comprised in the exhaled air can be conveniently collected without requiring difficult procedures for the person. Compared to sample collection through a nasal or throat swab, or saliva collection, the sample collector provides a particle collection with much less discomfort for the user and may remove the need for a trained professional to be involved in the sample collection.

The sample collector may thanks to the inlet for receiving a flow of air provide an easy and intuitively way of collecting samples through self-test by persons, thereby promote more frequent testing of people. In this regard, the use of the sample collector for screening of people may allow people to be screened several times per week, such as every day, such that viral load peaks may be identified. If testing is done only once a week, the viral load peak may be missed such that the testing fails to detect persons having a stage of a disease in which the person is highly infectious. Hence, very frequent testing may be required in order to identify highly infectious people and prevent spreading of a disease.

The particle capturing substrate enables an efficient capturing of the airborne particles, ensuring that the particles may be prepared for analysis e.g. for determining presence of a substance. As the same particle capturing substrate can be used for sampling and analysis, there is no need to move the collected particles from one substrate to another. The collected particles may even undergo one or more steps of reactions while being arranged on the same particle capturing substrate before analysis is carried out. Compared to other substrates for sample collection, not suitable for analysis such as a swab or the inside of a vessel, using one and the same particle capturing substrate for all steps may provide for testing in fewer process steps and facilitate a fast analysis.

Thanks to the sample collector being reconfigurable from the sampling configuration to an analysis configuration, the need for removing the particle capturing substrate from the sample collector during analysis may be eliminated thus ensuring safe handling, and facilitating a fast analysis of the sample. As there is a possibility for the sample carrying an infectious disease, a safe handling inside one and the same sample collector reduces the risk of contamination from the collected sample.

The analysis of the sample is facilitated by the aperture and the optical window providing optical access to the particle capturing substrate when arranged in the analysis compartment. As may be appreciated, optical access enables optical analysis methods, such as light-based measurements to be performed on particles captured by the particle capturing substrate.

By the particle capturing substrate being clamped by the substrate clamping device (hereinafter abbreviated “clamping device”) between the support member and the optical window while in the analysis configuration, a substrate-sealing function is provided. More specifically, the support member and the optical window may press against the particle capturing substrate from mutually opposite sides (i.e. main surfaces) of the particle capturing substrate to cover air inlets and outlets provided thereon. The clamping thus allows the particle capturing substrate to be “sealed” such that particles captured by the particle capturing substrate may be confined to the particle capturing substrate. This may further contribute to a safe handling and a reduced risk of spreading potentially hazardous captured particles.

The clamping function may further facilitate an efficient analysis with minimized optical artifacts by allowing the optical window to be pressed tightly against the particle capturing substrate, reducing presence of air gaps between the optical window and the particle capturing substrate.

The particle capturing substrate may be pressed between the support member and the optical window at a certain pressure level. As an example, a pressure level of 1 bar or more may counteract spreading of captured particles and leakage and transpiring of liquid from the substrate at the possibly elevated temperatures involved in various types of analysis. However, increasing the pressure may provide a further safety margin and facilitate more robustness in the device.

During the reconfiguration to the analysis configuration, the clamping device and the particle capturing substrate are configured to be moved together to (e.g. towards and into) the analysis compartment while the clamping device is brought (gradually) from an open state to the closed state. In other words, during the reconfiguration to the analysis configuration, the clamping device and the particle capturing substrate are configured to be moved together to the analysis compartment wherein the sample collector is configured to bring the clamping device to the closed state while the clamping device and the particle capturing substrate are moved together to the analysis compartment. This implies that the clamping of the particle capturing substrate may be obtained merely by moving the particle capturing substrate together towards and into the analysis compartment. This may facilitate handling of the sample collector, in that the reconfiguration and clamping (and hence sealing-function) need not involve any complex user interaction.

As used herein, a “closed state” of the clamping device refers to a state wherein the optical window and the support member are pressed against opposite sides of the particle capturing substrate such that the particle capturing substrate is clamped.

Conversely, an “open state” of the clamping device refers to a state wherein the optical window and the support member are spaced apart such that the particle capturing substrate may be received by the clamping device. The particle capturing substrate may hence be arranged with a clearance fit between the optical window and the support member.

According to embodiments, the substrate clamping device and the particle capturing substrate may be configured to, during reconfiguration from the sampling configuration to the analysis configuration, be moved together in a sliding movement along first and second guide surfaces (of the sample collector) configured to engage with the optical window and the support member during the sliding movement to press the optical window and the support member towards each other.

Hence, the clamping device may be brought gradually to the closed state from the open state by means of a simple mechanism.

The first and second guide surfaces may be arranged in front of the analysis compartment (e.g. as seen in a direction from the sampling compartment).

The first and second guide surfaces may be mutually opposite guide surfaces.

The first and second guide surfaces may be arranged to converge along a direction towards the analysis compartment.

The optical window and the support member may be gradually pressed towards each other by the converging first and second guide surfaces during a sliding movement towards the analysis compartment. Hence, a gradual closing of the clamping device may be facilitated.

The first and second guide surfaces may be provided by a pair of interior wall portions of a housing of the sample collector.

According to embodiments, the sample collector may comprise a housing wherein the sampling compartment and the analysis compartment are defined within the housing. The aperture at the analysis compartment may be formed in a wall portion of the housing. The housing may form an enclosure of the sample collector.

According to embodiments, the sample collector may comprise a substrate holder configured to hold the particle capturing substrate and configured to be movable such that the particle capturing substrate is movable, together with the clamping device, to the analysis compartment.

The sample collector may comprise a gripping part configured to be accessible from an exterior of the sample collector and coupled to the substrate holder such that the substrate holder may be moved by movement of the gripping part. Consequently, the reconfiguration to the analysis configuration may be facilitated by movement of the gripping part.

According to embodiments wherein the sample collector comprises a housing, the gripping part may be accessible from an exterior of the housing, e.g. by protruding outside the housing.

According to embodiments, the optical window and the support member may be pivotably connected to each other. A pivotable connection enables a simple and effective construction of the clamping device.

The particle capturing substrate may comprise a set of air inlets and set of air outlets, the set of air inlets and the set of air outlets provided on opposite sides of the particle capturing substrate, wherein the optical window and the support member are configured to cover a respective one of the set of air inlets and the set of air outlets when the sample collector is in analysis configuration. The particle capturing substrate may accordingly be configured to capture particles from a flow of air passing through the particle capturing substrate. The set of air inlets may be provided on a first side of the particle capturing substrate and the set of air outlets may be provided on a second side of the particle capturing substrate, or vice versa. The particle capturing substrate may further comprise an interior particle capturing chamber between the air inlets and air outlets.

The particle capturing substrate may especially be an aerosol impactor substrate. An aerosol impactor substrate allows efficient capturing of aerosols, and hence of aerosol borne substances.

According to embodiments the sample collector may further comprise an additional compartment, wherein in the sampling configuration, the substrate clamping device is arranged in the additional compartment. Providing an additional compartment enables a simplified design of the sample collector, e.g. in that the sampling compartment need not accommodate the clamping device. Additionally, the clamping device need not interfere with the flow of air on its way to the particle capturing substrate. Moreover, exposing the clamping device to debris or moisture in the flow of air from the inlet may be avoided during sampling.

According to embodiments the sample collector may be reconfigurable from the sampling configuration to the analysis configuration via an intermediate configuration wherein the substrate clamping device is in an open state to receive the particle capturing substrate between the optical window and the support member.

In embodiments wherein the optical window and the support member are pivotably connected to each other, an opening side of the substrate clamping device may be arranged to face the sampling compartment. Thereby, the particle capturing substrate may be received by substrate clamping device via the opening side.

According to embodiments the additional compartment may be arranged intermediate the sampling compartment and the analysis compartment, and wherein, during reconfiguration from the sampling configuration to the intermediate configuration, the particle capturing substrate may be configured to be moved to the additional compartment to be received between the optical window and the support member, and thereafter be moved, together with the substrate clamping device, to the analysis compartment.

This may facilitate handling of the sample collector, in that the reconfiguration and clamping (and hence sealing-function) need not involve any complex user interaction. E.g. the reconfiguration may involve a simple movement of the particle capturing substrate from the sampling compartment, to the clamping device in the intermediate compartment, and subsequently a movement of the particle capturing substrate and the clamping device together to the analysis compartment.

According to embodiments, the substrate clamping device may instead be arranged in the sampling compartment when the sample collector is in the sampling configuration, wherein the particle capturing substrate is arranged between the optical window and the support member of the substrate clamping device, the substrate clamping device being in an open state such that passage of the flow of air through the particle capturing substrate is allowed.

This may allow, among others, a shorter movement path of the particle capturing substrate towards the analysis compartment, and accordingly a more compact sample collector.

The sample collector may comprise a sealing structure configured to further seal the particle capturing substrate when the sample collector is in the analysis configuration.

According to embodiments the substrate clamping device may comprise a first sealing structure arranged on and configured to seal the substrate clamping device when the sample collector is in the analysis configuration (e.g. in a direction towards the sampling compartment).

A sealing structure may further contribute to sealing the particle capturing substrate when arranged in the analysis compartment and further reduce a risk of spreading of captured particles and leakage and transpiring of liquid from the particle capturing substrate.

According to embodiments the first sealing structure may comprise a first sealing member arranged to extend along an entire circumference of the optical window. The first sealing structure may further comprise a second sealing member arranged to extend along an entire circumference of the support member.

Thereby, the first sealing structure may be configured to seal the clamping device when in the closed state and clamping the particle capturing substrate.

The terms “circumferentially” and “circumference” as used herein are not be construed as necessarily implying a circular shape of the element being “circumferentially enclosed”/having the “circumference”, but is also intended to encompass other shapes, such as rectangular or square shapes or other polygonal shape. Accordingly, a circumference may be considered synonymous to “perimeter”.

According to embodiments, the first sealing member may be configured to circumferentially enclose a (first) surface region of a first side of the particle capturing substrate in which a set of air inlets or outlets are provided, and the second sealing member may be configured to circumferentially enclose a (second) surface region of a second side of the particle capturing substrate in which a set of air outlets or inlets are provided, when the sample collector is in the analysis configuration.

A seal completely circumferentially enclosing the air inlets and air outlets of the particle capturing substrate may hence be provided when the particle capturing substrate is arranged in the analysis position.

If dedicated reagent inlets are provided on the first/second side of the particle capturing substrate, the first/second surface region enclosed by the first/second sealing member may further encompass any reagent inlet.

The first and second sealing member may be configured to engage with (mutually) opposite sides of a frame portion circumferentially enclosing the particle capturing substrate, when the sample collector is in the analysis configuration. The frame portion may be a frame portion of the aforementioned substrate holder configured to hold the particle capturing substrate.

According to embodiments the sealing members may be configured to define a respective sealed volume between the sealing member and a peripheral edge of the particle capturing substrate when the sample collector is in the analysis configuration.

The sealed volumes may form an expansion volume, e.g. for accommodating excess liquid (e.g. liquid reagent) from the particle capturing substrate.

According to embodiments the sample collector may further comprise a thermal body configured to, when the sample collector is in the sampling configuration, heat the flow of air received from the inlet prior and/or the particle capturing substrate. Heating by the thermal body may counteract condensation on or in the particle capturing substrate, e.g. of moisture present in the flow of air.

According to embodiments, the sample collector may further comprise a reagent feed for receiving reagent to be supplied to the particle capturing substrate.

By reagent is hereby meant a substance or mixture for use in chemical analysis or other reactions.

The reagent may be liquid.

According to embodiments, the particle capturing substrate may further be reconfigurable from the sampling configuration to a reagent filling configuration, wherein the particle capturing substrate is arranged to: be aligned with the reagent feed; and receive the reagent through the reagent feed.

The reagent feed may be provided at a position intermediate the sampling compartment and the analysis compartment. In embodiments comprising the additional compartment intermediate the sampling compartment and the analysis compartment, the reagent feed may be provided at a position intermediate the sampling compartment and the additional compartment. The reagent may hence be supplied to the particle capturing substrate prior to being received in the clamping device.

According to embodiments, the particle capturing substrate may comprise one or more reagent inlets, wherein, when in the reagent filling configuration, the one or more reagent inlets of the particle capturing substrate may be aligned with the reagent feed.

The reagent inlets may be provided on a first side or second side of the particle capturing substrate. The one or more reagent inlets may be connected to an interior particle capturing chamber of the particle capturing substrate, located between air inlets and air outlets of the particle capturing substrate.

Embodiments of the sample collector comprising a reagent feed may advantageously be combined with embodiments comprising a sealing structure, as set out above, wherein the sealing structure may counteract leakage and transpiration of reagent from the particle capturing substrate and the clamping device during analysis.

According to embodiments, the particle capturing substrate may be configured to capture the airborne particles and may further be configured to receive a reagent to be mixed with the collected particles, for preparation for thermal lysis to expose RNA in the collected particles The RNA may be converted to DNA using reverse transcriptase based on the reagent and, wherein the sample collector is configured to facilitate thermal cycling for amplification of the DNA using quantitative polymerase chain reaction. For example, the RNA of may be RNA of severe acute respiratory syndrome coronavirus 2 (SARS-COV-2).

According to a further aspect there is provided a kit of parts comprising:
a sample collector as set out above; and
an analysis instrument configured to receive the sample collector for optical analysis of the airborne particles captured by the particle capturing substrate,
wherein, when the sample collector is received in the analysis instrument and the particle capturing substrate is arranged in the analysis configuration,
optics of the analysis instrument is configured to be aligned with the optical window of the sample collector for optical access to the particle capturing substrate while the analysis instrument is configured to press the optical window towards the support member such that the particle capturing substrate is clamped therebetween.

The sample collector may hence after sampling be re-configured from the sampling configuration to the analysis configuration, and then transferred to the analysis instrument for optical analysis of the captured particles.

By the optics pressing the optical window towards the support member, a pressure/clamping force applied to the particle capturing substrate when arranged in the analysis configuration may be increased beyond that provided by the clamping by the optical window and the support member.

According to embodiments, the front surface of the optics may be configured to abut and press the optical window towards the support member.

According to embodiments, the analysis instrument may comprise a heat source, and the support member may be a thermal member configured to allow thermal energy to be supplied from the heat source of the analysis instrument to the particle capturing substrate, when the sample collector is received in the analysis instrument and the particle capturing substrate is arranged in the analysis position.

By the thermal surface being configured to allow thermal energy to be supplied from the heat source of the analysis instrument to the analysis compartment of the sample collector, heat-induced reactions are allowed on the particle capturing substrate.

According to a further aspect, there is provided a method for collection of airborne particles exhaled by a human being with (i.e. by) a sample collector as set out above, the method comprising:
receiving via the inlet of the sample collector a flow of air, the flow of air carrying the airborne particles;
capturing the airborne particles in the flow of air from the inlet by the particle capturing substrate while the sample collector is configured in the sampling configuration; and
reconfiguring (e.g. subsequent to the step of capturing) the sample collector to the analysis configuration wherein the substrate clamping device and the particle capturing substrate are arranged in the analysis compartment, the optical window is aligned with the aperture such that optical access to the particle capturing substrate is allowed, and the substrate clamping device is in the closed state such that the particle capturing substrate is clamped between the optical window and the support member;
   wherein the reconfiguring step comprises moving the substrate clamping device and the particle capturing substrate together to the analysis compartment while the substrate clamping device is brought to the closed state.

According to embodiments the reconfiguring step may comprise moving the substrate clamping device and the particle capturing substrate together in a sliding movement along first and second guide surfaces of the sample collector configured to engage with the optical window and the support member during the sliding movement to press the optical window and the support member towards each other.

According to embodiments the reconfiguring step may comprise moving the particle capturing substrate from the sampling compartment to the afore-mentioned additional compartment to be received between the optical window and the support member, and thereafter moving the substrate clamping device and the particle capturing substrate together to the analysis compartment.

According to embodiments wherein the clamping device is arranged in the sampling compartment in the sampling configuration as set out above, the reconfiguring step may comprise moving the substrate clamping device and the particle capturing substrate together from the sampling compartment to the analysis compartment.

According to embodiments, the method may further comprise, while the sample collector is in the sampling configuration, heating the flow of air received from the inlet prior and/or the particle capturing substrate using a thermal body.

According to embodiments, the method may further comprise comprising supplying a reagent to the particle capturing substrate by introducing a reagent through a reagent feed of the sample collector.

According to embodiments, the method may further comprise, subsequent to the step of capturing, reconfiguring the sample collector from the sampling configuration to a reagent filling configuration, and subsequent to the step of supplying the reagent, reconfiguring the sample collector to the analysis configuration.

According to embodiments the step of capturing may comprise capturing the airborne particles, and the step of introducing may comprise a reagent to be mixed with the collected particles, for preparation for thermal lysis to expose RNA in the collected particles. The RNA may be converted to DNA using reverse transcriptase based on the reagent and providing thermal cycling for amplification of the DNA using quantitative polymerase chain reaction. For example, the RNA of may be RNA of severe acute respiratory syndrome coronavirus 2 (SARS-COV-2).

As the method provides an easy and intuitively way of collecting samples through self-test by persons, it enables high-volume screening of people for infectious diseases such as coronavirus disease 2019 (COVID-19).

### BRIEF DESCRIPTION OF THE DRAWINGS

The above, as well as additional objects, features, and advantages, may be better understood through the following illustrative and non-limiting detailed description, with reference to the appended drawings. In the drawings like reference numerals will be used for like elements unless stated otherwise.

FIG. 1 is a schematic perspective cross-sectional view of a sample collector according to an embodiment.

FIG. 2 shows the sample collector in a sampling configuration.

FIG. 3 shows the sample collector in a reagent filling configuration.

FIG. 4 shows the sample collector in an intermediate configuration.

FIG. 5 shows a zoomed-in view of an analysis compartment when the sample collector is in an analysis configuration.

FIG. 6 shows in perspective an exterior of the sample collector.

FIG. 7 is a schematic perspective cross-sectional view of a sample collector according to a further embodiment.

FIG. 8 is a schematic perspective cross-sectional view of a sample collector according to a further embodiment.

FIG. 9 is a schematic view of a kit of parts according to an embodiment.

### DETAILED DESCRIPTION

In the following, embodiments of a sample collector suitable for collection of airborne particles comprised in exhaled air by a test subject (a human being) will be disclosed. The sample collector may be used to facilitate determining whether a person carries a disease, which is spread through droplets and/or aerosols produced during breathing. For instance, the sample collector may be used for screening whether a person is infected by influenza or severe acute respiratory syndrome coronavirus 2 (SARS-COV-2). However, it is to be noted that a sample collector according to the present invention is suitable for also in other applications relying on collection of airborne particles exhaled by a human being, such as droplet or aerosol borne chemical substances.

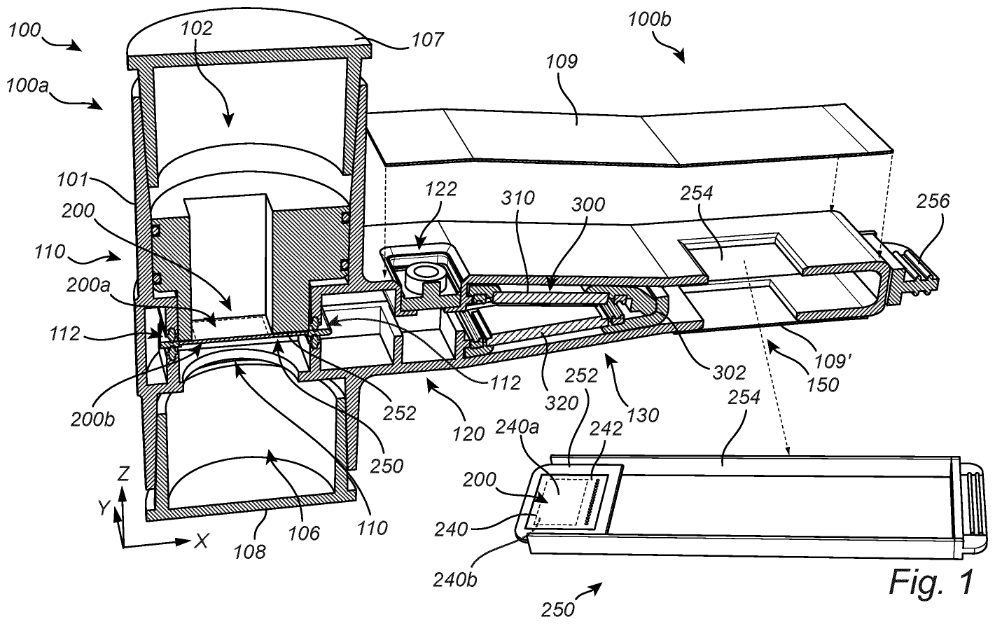

Referring now to FIGS. 1-5, a sample collector 100 for collection of airborne particles exhaled by a human being will be described.

The sample collector 100 comprises a housing 101 forming an enclosure of the sample collector 100. The housing 101 may as shown be formed as an elongated body with a substantially rectangular hollow cross section. Reference signs 100a and 100b respectively designate a front portion and a rear portion of the sample collector 100. The housing may e.g. be formed by a suitable plastic material.

In the figures, axes X, Y, Z correspond to respectively a length dimension, a width dimension and a height dimension of the sample collector 100 and housing 101. The cross-section may extend along a mid-line of the sample collector 100. The sample collector 100 may be symmetrical about the mid-line such that the cut-away part of the sample collector 100 is identical to the illustrated part, albeit mirrored.

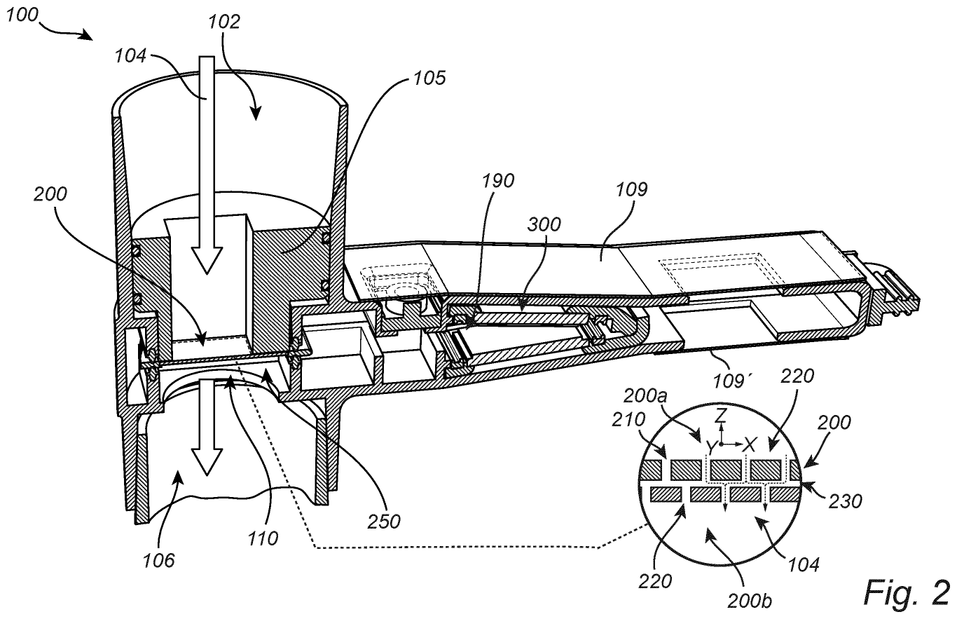

The sample collector 100 comprises an inlet 102 for receiving a flow of air 104 (indicated in FIG. 2). The inlet 102 is provided in the front portion 100a of the sample collector 100. The flow of air 104 may be received from exhalation by the test subject. A mouthpiece may be connected to the inlet 102 to facilitate exhalation into the sample collector 100. The mouthpiece may be inserted into the mouth of the test subject who may exhale through the mouthpiece to provide the flow of air 104 into the inlet 102. It is envisaged that a mouthpiece may be integrally formed with the inlet or separately formed and connected thereto, fixedly or in a detachable manner. According to other embodiments a mouthpiece may be omitted. For example, the inlet 102 may be adapted to interface with a container, e.g. in the form of a breathing bag or the like, into which the test subject has exhaled. The flow of air 104 may hence enter the inlet 102 from the container, e.g. by collapsing the breathing bag.

The sample collector 100 further comprises a sampling compartment 110 arranged to receive the flow of air 104 from the inlet 102, as further described below. The sampling compartment 110 is arranged within the housing 101. The sampling compartment 110 is arranged in the front portion 100a of the sample collector 100.

The sample collector 100 further comprises an outlet 106 allowing the flow of air 104 to exit the sample collector 100 after passing through a sampling compartment 110, to be described in the following. The outlet 106 is arranged in the front portion 100a of the sample collector 100. The outlet 106 may be preceded by an air-permeable filter, to reduce a risk of ejecting particles not captured by the particle capturing substrate 200 from the sample collector 100.

For improved safety, the inlet 102 as well as the outlet 106 may be closed by a respective cap or plug 107, 108, or alternatively by a sealing tape or the like, prior to sampling and after sampling, as indicated in FIG. 1.

Reference signs 109 and 109′ indicate sealing tapes which may be used to cover reagent inlets and/or apertures of the sample collector 100 before and/or after sampling.

The sample collector 100 further comprises a particle capturing substrate 200. The particle capturing substrate 200 is configured to capture airborne particles in the flow of air 104 passing through the particle capturing substrate 200. As will be further described herein with reference to FIG. 2, the sample collector 100 has a sampling configuration wherein the particle capturing substrate 200 is arranged in the sampling compartment 110 to receive the flow of air 104 from the inlet 102.

The particle capturing substrate 200 has a first side 200a and an opposite second side 200b, forming a front side and a rear side, as seen in a direction from the inlet 102 towards the outlet 106. The particle capturing substrate 200 is depicted with a substantially rectangular shaped outline. It should however be noted that also other shapes are possible such as a rounded shape. The particle capturing substrate 200 may be oriented such that the plane of extension is parallel with the XY-plane of the sample collector 100. Hence, the term “in-plane” or “in-plane direction” with respect to the particle capturing substrate 200 may be understood as a plane or direction parallel to the XY-plane.

For illustrative clarity the particle capturing substrate 200 is in FIG. 1 laid bare outside the sample collector 100, together with a sample holder 250 described below. Reference sign 240 commonly designates a circumference/perimeter of an air inlet/outlet region 240a of the first side 200a of the particle capturing substrate 200, and of an inlet/outlet region 240b of the second side 200a of the particle capturing substrate 200. The air inlet/outlet regions are to be understood as the regions in which the air inlets or air outlets respectively are disposed. Reference sign 242 designates a peripheral surface region or frame region of the particle capturing substrate 200, circumferentially enclosing the air inlet/outlet region 240.

The inset in FIG. 2 schematically illustrates a zoomed in cross-section of a portion of the particle capturing substrate 200, according to an embodiment. The cross-section may be representative for a cut taken along either the X direction or the Y direction. As shown, the particle capturing substrate 200 may comprise a set of air inlets 210 and a set of air outlets 220 provided on an air inlet side (e.g. the first side 200a) and an air outlet side (e.g. the second side 200b). The air inlets 210 and the air outlets 220 may be distributed within a sub-region of the air inlet side and the air outlet side, respectively. The extension of the sub-regions may correspond to a major portion of the air inlet side and the air outlet side but may also be smaller.

The example flow of air 104 indicates that the air inlet side is defined by the first side 200a of the particle capturing substrate 200 while the air outlet side is defined by the second side 200b of the particle capturing substrate 200. The air inlet side may alternatively be defined by the second side 200b of the particle capturing substrate 200 and the air outlet side may be defined by the first side 200a of the particle capturing substrate 200, wherein the sampling compartment 110 may be configured to guide the flow of air 104 in the opposite direction through the particle capturing substrate 200.

The particle capturing substrate 200 may as further shown comprise an interior particle capturing chamber 230 between the air inlets and air outlets. The air inlets 210 and air outlets 220 may be displaced with respect to each other along the in-plane directions X/Y such that the air inlets 210 will face a wall portion of the particle capturing chamber 230 between outlets 220 such that particles carried by the flow of air 104 passing through the particle capturing substrate 200 may be captured at least on wall portions of the particle capturing chamber 230 by impaction. Thus, the air inlets 210 and air outlets 220 may be staggered such that the central axes of the air inlets 210 and the central axes of the air outlets 220 are not aligned.

As an example, a diameter of a circular cross-section of the air inlets 210 may be in a range of 20-300 μm, such as in a range of 100-200 μm. The size and shape of the air inlets 210 may be chosen in relation to at least a desired efficiency of collection of particles, a desired size of particles to be collected, and a pressure drop to be experienced by the flow of air 104. A large number of air inlets 210 may be used in order to allow a large total flow rate through the air inlets 210 while the air inlets 210 have a small cross-section. For instance, the number of air inlets 210 may be larger than 100, such as larger than 500, such as 1000-2000 air inlets 210. The size and shape of the air outlets 220 may be chosen in relation to the size and shape of the air inlets 210 for providing a staggered relationship between the air inlets 210 and the air outlets 220. As a further example, a diameter of a circular cross-section of the air outlets 220 is in a range of 20-400 μm, such as in a range of 100-300 μm. Since the dimension of the air outlets 220 may not directly influence the collection efficiency of the particle capturing substrate 200, the air outlets 220 may in some embodiments be larger than the air inlets 210. The air inlets 210 and/or the air outlets 220 may be tapered towards a smaller cross-section.

It should further be realized that a shape of the cross-section of the air inlets 210 and/or the air outlets 220 need not be circular. Rather, the air inlets 210 and/or the air outlets 220 may be square, elliptical or rectangular instead. In particular, the cross-sections of the air inlets 210 and the air outlets 220 may have a shape of a rectangular slit. In such case, the air inlets 210 may have a large length and a small width, wherein the width is in the range of 20-300 μm, such as in the range of 100-200 μm for ensuring efficient particle collection.

The particle capturing substrate 200 may be an aerosol impactor substrate, implying that the density, dimensions and layout of the inlets 210, outlets 220 and the particle capturing chamber 230 are such that aerosols may be captured in the particle capturing chamber through impaction.

If collection of small droplets, such as droplets down to a size of 300 nm is not important, a larger size of the inlet diameters may be accepted.

The particle capturing substrate 200 may be formed from a semiconductor or semiconductor-based material, such as layers of silicon and/or silicon dioxide. Fabrication of the particle capturing substrate 200 may comprise using microfabrication techniques, such as etching inlets 210 in a first layer, outlets in a second layer 220, and a particle capturing chamber 230 in a third layer and subsequently bonding the first and second layers to opposite sides of the third layer.

The sample collector 100 further comprises a thermal body 105 arranged in the sampling compartment 110. The thermal body 105 is configured to heat the flow of air 104 received from the inlet 102 prior to reaching the particle capturing substrate 200, during sampling. The thermal body 105 may comprise a channel allowing the flow of air 104 from the inlet 102 to the particle capturing substrate 200. The thermal body 105 may be formed by thermally conductive material such as a metal. The thermal body 105 may be exposed from an exterior of the sample collector 100 by an opening in the housing 101 such that the thermal body 105 may be brought into contact with a heating device. The thermal body 105 may be movable from a position in abutment with the particle capturing substrate 200 (“abutting position”), to a position removed from the particle capturing substrate 200 (“removed position”). The thermal body 105 may be arranged in the abutting position during sampling, and be moved to the removed position prior to moving the particle capturing substrate 200 from the sampling compartment 110. One or more sealing gaskets may as indicated in FIG. 1 be arranged to seal an interface between the thermal body 105 and an interior wall portion of the housing 101.

The sample collector 100 further comprises an analysis compartment 150. The analysis compartment 150 is arranged within the housing 101. The analysis compartment 150 is provided in the rear portion 100b of the sample collector 100. An aperture 152 is defined in a wall portion of the housing 101 to provide optical access to an interior of the analysis compartment 150.

The sample collector 100 further comprises an additional compartment 130. The additional compartment 130 is arranged in a portion of the housing intermediate the front portion 100a and the rear portion 100b, as viewed along the length dimension X. More specifically, the additional compartment 130 is arranged intermediate the sampling compartment 110 and the analysis compartment 150. The additional compartment 130 may hence be referred to as an intermediate compartment 130.

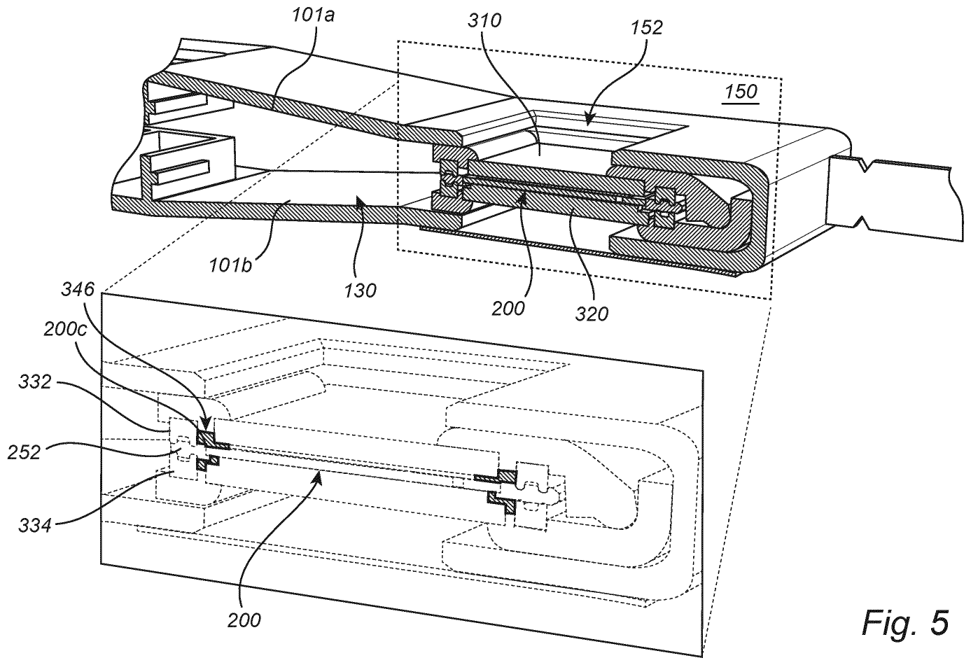

The sample collector 100 further comprises a substrate clamping device 300 comprising an optical window 310 and a support member 320. The clamping device 300 is re-configurable between an open state (see e.g. FIGS. 1-4) for receiving the particle capturing substrate 200 between the optical window 310 and the support member 320, and a closed state (see e.g. FIG. 5) for clamping the particle capturing substrate 300 between the optical window 310 and the support member 320.

The optical window 310 and the support member 320 are pivotably connected to each other. The clamping device 300 has an opening side located opposite the side of the pivotable connection. The opening side faces towards the sampling compartment 110 for receiving the particle capturing substrate 200. The pivotable connection may as shown be provided by a hinge device 302. The hinge device 302 may be biased (e.g. spring-loaded) to maintain the clamping device 300 in the open state. Alternatively, the optical window 310 and the support member 320 may be pivotably connected by means of a resilient member (e.g. of a suitably elastic/resilient plastic or rubber material) in place of the hinge device 302.

The optical window 310 is configured to provide optical access to the particle capturing substrate 200, when received in the clamping device 300.

Optical access to the particle capturing substrate 200 enables light-based measurements of the airborne particles collected as samples by the particle capturing substrate 200. This enables the particle capturing substrate 200 to be contained in the sample collector 100 throughout the whole process from sampling to analysis.

The optical window 310 may be formed by a transparent or translucent material, which allows light to be passed through. Alternatively, the light-based measurement may be configured to image particles in the analysis compartment or relate detected light to a position in the analysis compartment. In such case, the optical window may be formed by a transparent material to allow light to pass mostly unaffected through the optical window.

It should further be realized that the optical window 310 need not necessarily be transparent or translucent in an entire area related to the analysis compartment. Rather, the optical window 310 may comprise portions that are transparent or translucent.

The support member 320 may e.g. be formed by a thermally conductive material such as a metal to facilitate temperature control of the particle capturing substrate 200 during analysis by means of an external heat source, as will be further discussed below. Alternatively, if temperature control is not needed during analysis (or if temperature control may be provided by other means such as indirect radiative heating) the support member 320 may be formed by e.g. a plastic material or the like.

The optical window 310 and the support member 320 may each be supported by a respective frame member 312, 322, circumferentially enclosing the optical window 310/support member 320. The frame members may be attached to the hinge device 310.

The particle capturing substrate 200 is configured to be movable from the sampling compartment 110 (a sampling position of the particle capturing substrate 200) to the analysis compartment 150 (an analysis position of the particle capturing substrate 200).

To facilitate handling and movement of the particle capturing substrate 200, the particle capturing substrate 200 may be arranged on a substrate holder 250, fully exposed in FIG. 1. The substrate holder 250 may comprise an opening enclosed by a frame portion 252 and in which the particle capturing substrate 200 may be received and supported.

The substrate holder 250 is configured to be movable, relative the analysis compartment 150, such that the particle capturing substrate 200 is movable from the sampling compartment 110 to the analysis compartment 150. The substrate holder 250 may be configured to move in a sliding in-plane movement through the housing 101 (along the X dimension). The substrate holder 250 may be configured to bear slidingly against guide surfaces formed by interior partition walls of the housing 101 and/or sealing structures 112, to be guided during the movement.

A gripping part 256 in the form of a tab or handle is provided on the substrate holder 250 to be accessible from an exterior of the sample collector 100. The gripping part 256 protrudes at the rear portion 100a of sample collector 100. The gripping part 256 is connected to the frame portion 252 by an elongated coupling member 254. The coupling member 254 may be arranged to extend from the frame portion 254 towards the gripping part 256, past the clamping device 300, laterally with respect to the clamping device 300. The coupling member 254 may as shown be formed with a vertically oriented rectangular oblong shape (parallel to the XZ-plane) and extend along a sidewall of the housing 101, to not interfere with e.g. the clamping device 300. A corresponding coupling member 254 may as shown in the inset be provided to extend along the mutually opposite sidewall of the housing 101.

The coupling member(s) 254 may protrude through a (respective) slit formed in the housing 101 at the rear portion 100a, to connect to the gripping part 256. A sealing gasket may be arranged in the (respective) slit to provide sealing between the housing 101 and the coupling member(s) 254.

The substrate holder 250 and the particle capturing substrate 200 may accordingly be moved towards the analysis compartment 150 by pulling the gripping part 256.

In the illustrated embodiment, the gripping part 256 is integrally formed with the substrate holder 250. However, it is also possible to form the gripping part 256 as a separate structure, to be fastened to the coupling member(s) 254, e.g. by gluing, welding or the like. Additionally, the depicted shape of the gripping part 256 is merely an example and other shapes allowing a secure gripping may be used.

FIG. 2 shows the sample collector in a sampling configuration. The caps 107, 108 have been removed to allow the inlet 102 to receive a flow of air 104 and allow the flow of air 104 to exit via the outlet 106 after passage through the particle capturing substrate 200.

The particle capturing substrate 200 is arranged in the sampling compartment 110 to receive a flow of air 104 from the inlet 102 and accordingly capture airborne particles in the flow of air 104. The thermal body 105 is arranged in the abutting position to heat the flow of air 104 passing therethrough. The clamping device 300 is arranged in the additional compartment 130 in the open state.

As shown in FIG. 2, the sample collector 100 is configured to guide the flow of air 104 from the inlet 102 such that the flow of air 104 passes through the particle capturing substrate 200. The flow of air 104 is guided to enter the particle capturing substrate 200 from an air inlet side (e.g. 200a) thereof, pass through the particle capturing substrate 200, and exit the particle capturing substrate 200 from an air outlet side thereof (e.g. 200b). Alternatively, direction of the flow of air 104 may be reversed such that the air inlet side of the particle capturing substrate 200 may be defined by the second side 200b and the air outlet side may be defined by the first side 200a of the particle capturing substrate 200. For example, the roles of the inlet 102 and the outlet 106 may be exchanged.

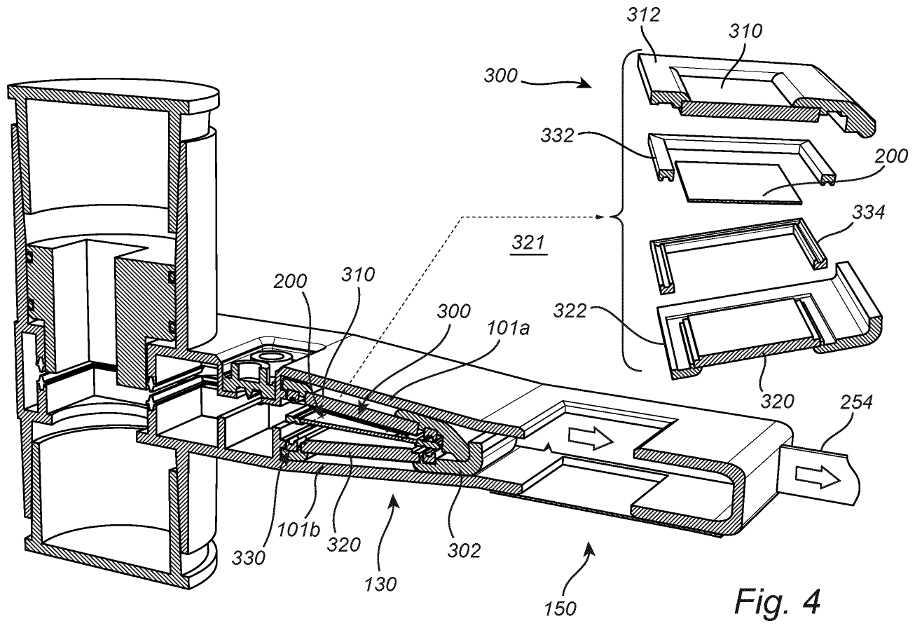

FIG. 4 shows the sample collector after re-configuration into an intermediate configuration. The particle capturing substrate 200 has been moved to the additional compartment 130 to be received between the optical window 310 and the support member 320 of the clamping device 300 (being in the open state). The movement is facilitated by actuation of the gripping part 256, e.g. by pulling the gripping part 256 along the length dimension X. As indicated in FIG. 4, the inlet 102 and the outlet 106 may be resealed after sampling, e.g. by reinstalling the caps 107, 108, prior to initiating the re-configuration from the sampling configuration.

FIG. 5 shows the analysis compartment 150 after re-configuration from the intermediate configuration into analysis configuration. The particle capturing substrate 200 has been moved together with the particle capturing substrate 300 to the analysis compartment 150. The movement is facilitated by a further actuation of the gripping part 256, e.g. by pulling the gripping part 256 along the length dimension X.

In the analysis configuration, the substrate clamping device 300 and the particle capturing substrate 200 are arranged in the analysis compartment 150 such that the optical window 310 is aligned with the aperture 152 to allow optical access to the particle capturing substrate 200 via the optical window 310. The clamping device 300 is in a closed state such that the particle capturing substrate 200 is clamped between the optical window 310 and the support member 320.

The movement of the clamping device 300 and the particle capturing substrate 200 together towards and into the analysis compartment 150 causes a gradual bringing of the clamping device 300 to the closed state (i.e. a gradual closing of the clamping device 300). The sample collector 100 is configured to transfer the (linear) movement of the clamping device 300 and the particle capturing substrate 200 towards the analysis compartment, to a gradual closing of the clamping device 300.

According to the illustrated embodiment, the gradual closing of the clamping device 300 is facilitated by a pair of first and second guide surfaces 101a, 101b, arranged oppositely with respect to each other, and converging along a direction towards the analysis compartment 150.

The optical window 310 and the support member 320 are configured to bear slidingly against the first and second guide surfaces 101a, 101b, during the sliding movement towards the analysis compartment 150 wherein the optical window 310 and the support member 320 are gradually pressed towards each other by the first and second converging guide surfaces 101, 101b.

In the sample collector 100, the first and second converging guide surfaces 101a, 101b are defined by inwardly facing surfaces of the housing 101. Hence, the first and second converging guide surfaces 101a, 101b determine the outer dimensions of the housing 101. However, first and second converging guide surfaces may according to other embodiments be formed by interior wall portions or ledges, separate from the (outer) wall portions of the housing. In this case, the housing 101 may be formed with a constant cross-section from the additional compartment 130 to the analysis compartment 150. According to still further embodiments, the gradual closing may be provided by step-shaped first and second guide surfaces. For example, the housing 101 may be formed with a constant cross-section along the additional compartment 130 which abruptly changes to the cross-section of the analysis compartment 150.

In the analysis configuration of the sample collector 100, the particle capturing substrate 200 is clamped, i.e. press-fitted, between the optical window 310 and the support member 320. The press-fit/clamping implies that a thickness (i.e. along the height dimension Z) of the clamping device 300, with the particle capturing substrate 200, exceeds a height dimension of the analysis compartment 150 (i.e. along the height dimension Z) when the analysis compartment 150 is empty (i.e. absent from the clamping device 300 and the particle capturing substrate 200).

The press-fit/clamping of the particle capturing substrate 200 allows the particle capturing substrate 200 to be sealed when arranged in the analysis compartment 150, such that particles captured by the particle capturing substrate 200 may be confined thereto.

A respective area of optical window 310 and the support member 320 may be such as to completely cover the air inlet side (e.g. 200a) and the air outlet side (e.g. 200b) of the particle capturing substrate 200, or at least their respective air inlet/outlet region 240.

The sealing pressure applied to the particle capturing substrate 200 may as an example be at least 1 bar, at least 2 bar, or at least 3 bar. Such pressure levels may to an increasing extent enable a reliable sealing of the air inlets 210 and air outlets 220 of the particle capturing substrate 200, for example counteracting spreading of captured particles and transpiring of liquid. This may be particularly advantageous in testing applications involving a liquid reagent and heating during analysis, as will be further discussed below.

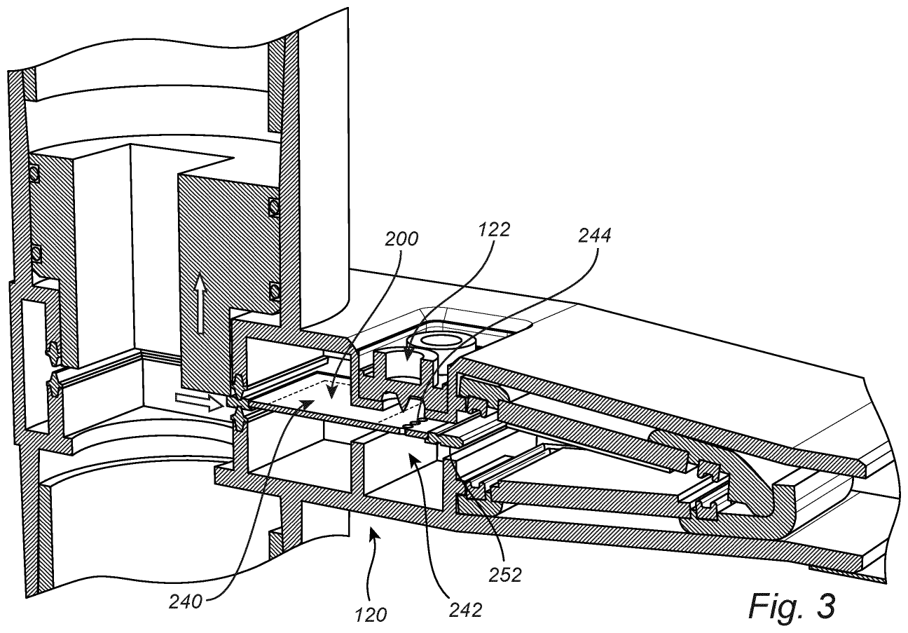

The sample collector 100 may further be re-configured into a reagent filling configuration, as shown in FIG. 3. The sample collector 100 comprises one or more reagent feeds or reagent inlets 122 for receiving reagent to be supplied to the particle capturing substrate 200. The particle capturing substrate 200 may as shown comprise one or more reagent inlets 244. The reagent inlet(s) may e.g. be provided in the peripheral surface region 242 of the particle capturing substrate 200. The reagent feed 122 may be provided at a reagent filling compartment 120 between the sampling compartment 110 and the additional compartment 130. The sample collector 100 may be re-configured from the sampling configuration to the reagent filling configuration by moving the particle capturing substrate 200 from the sampling compartment 110 such that the reagent inlet(s) 244 align with the reagent feed 122 to receive the reagent therefrom (i.e. a reagent filling position of the particle capturing substrate 200).

The reagent may be a substance or mixture, e.g. typically in liquid form such as solution, which may facilitate the subsequent analysis of the captured particles, as will be further discussed below.

The reagent inlet(s) 244 may communicate with an interior of the particle capturing substrate 200, e.g. the particle capturing chamber 230. The distribution of reagent within the particle capturing substrate 200 may be facilitated by capillary forces.

In addition to or as an alternative to dedicated reagent inlets 202, the one or more reagent feeds 122 may be configured to align with one or more air inlets 220 (or the air outlets 210, as the case may be, depending on the orientation of the particle capturing substrate 200 with respect to reagent feed(s) 140), wherein the reagent may be supplied to the air inlet(s) or outlet(s) 210, 220.

In either case, during the further movement towards the analysis compartment 150, the gradual closing of the clamping device 300 may contribute to spreading the reagent over the first side of the particle capturing substrate 200, to enter (further) air inlet(s) or outlet(s) 210, 220.

The above-discussed press-fitting/clamping of the particle capturing substrate 200 provided by the clamping device 300 may counteract leakage and/or transpiring of the reagent from the particle capturing substrate 200. The clamping may further counteract bubbles in the reagent by reducing a presence of bubbles at the interface between the optical window 310 and the particle capturing substrate 200.

The sample collector 100 may as further shown in FIGS. 1-5 comprise a sealing structure 330 (first sealing structure 330) configured to seal the clamping device 300 when the sample collector 100 is in the analysis configuration. This may further reduce a risk of spreading of captured particles and leakage and transpiring of liquid from the particle capturing substrate 200 and the sampling compartment 110, in the analysis configuration 330.

The sealing structure 330 is clearly shown in the exploded view of FIG. 4. The sealing structure 330 comprises a first sealing member 332 arranged to extend along an entire circumference of the optical window 310. The sealing structure 330 further comprises a second sealing member 334 arranged to extend along an entire circumference of the support member 320.

The first and second sealing members 332, 334 may as shown be separately formed sealing ridges or sealing gaskets, attached to the respective frame member 312, 322 of the clamping device 300, e.g. by being press-fitted in a receiving track formed in the respective frame member 312, 322 and/or attached by gluing or similar. It is however contemplated that the sealing members 332, 334 instead may be integrally formed with the respective frame members 312, 322.

The first and second sealing members 332, 334 are configured to engage with mutually opposite sides of the frame portion 252 of the substrate holder 250, when the sample collector 100 is in the analysis configuration, as is illustrated in FIG. 5. The first sealing member 332 may accordingly completely circumferentially enclose the air inlet/outlet region 240a of the first side 200a of the particle capturing substrate 200 (visible e.g. in FIGS. 1 and 2). The second sealing member 334 may correspondingly completely circumferentially enclose the air inlet/outlet region 240b of the second side 200b of the particle capturing substrate 200. The sealing structure 300 may thus provide a seal completely circumferentially enclosing the air inlets and air outlets 210, 220 of the particle capturing substrate 200. As the frame portion 252 extends along the entire circumference of the particle capturing substrate 200, the sealing structure 300 may also seal any reagent inlet(s) 244 of the particle capturing substrate 200. Additionally, the sealing members 332, 334 may as indicated in the zoomed in view of FIG. 5 define a respective sealed volume 346 between the respective sealing member 332, 334 and a peripheral edge 200c of the particle capturing substrate 200. The sealed volumes 346 may form an expansion volume, e.g. for accommodating excess liquid (e.g. liquid reagent) from the particle capturing substrate 200.

However according to an alternative embodiment, the sealing structure 330 may instead comprise first and second sealing members configured to engage with mutually opposite sides of the peripheral surface region 242 of the particle capturing substrate 200. In case reagent inlets 244 are provided in the peripheral surface region 242, the first and second sealing members may be configured to extend outside the reagent inlets 244 such that both the air inlet/outlet region 240a and the surface region comprising the reagent inlet(s) 244 are circumferentially enclosed.

As shown in FIG. 1, the sample collector 100 may comprise additional sealing structures, such as sealing structures 112 arranged in the sampling compartment 110, e.g. to avoid air leakage into other compartments of the sample collector 100.

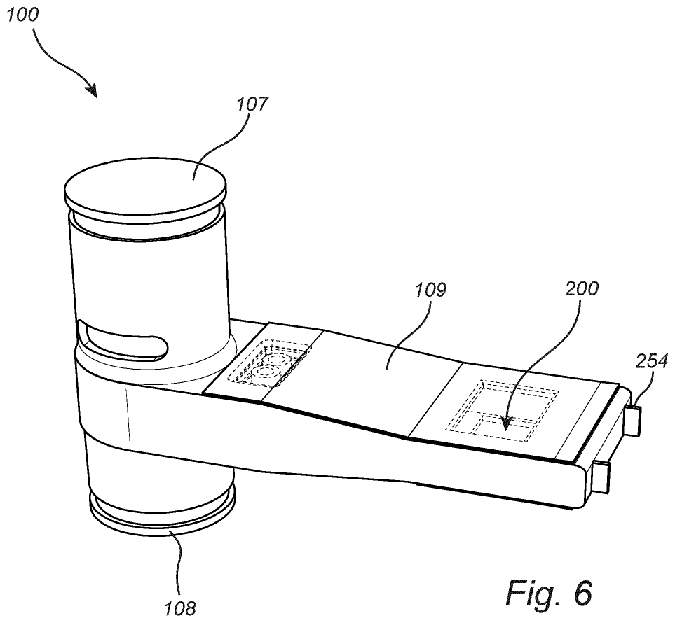

FIG. 6 is an exterior view of the sample collector 100 in the analysis configuration, wherein the inlet 102 and the outlet 106 are sealed. Additionally, a sealing type 109 has been attached to cover e.g. the reagent inlet 122 and the aperture 254. Additionally, the gripping part 256 has been broken off from the substrate holder 250, along with excess length of the coupling member(s) 254. The sample collector 100 may subsequently be transferred to an analysis instrument for optical analysis of the airborne particles captured by the particle capturing substrate 200.

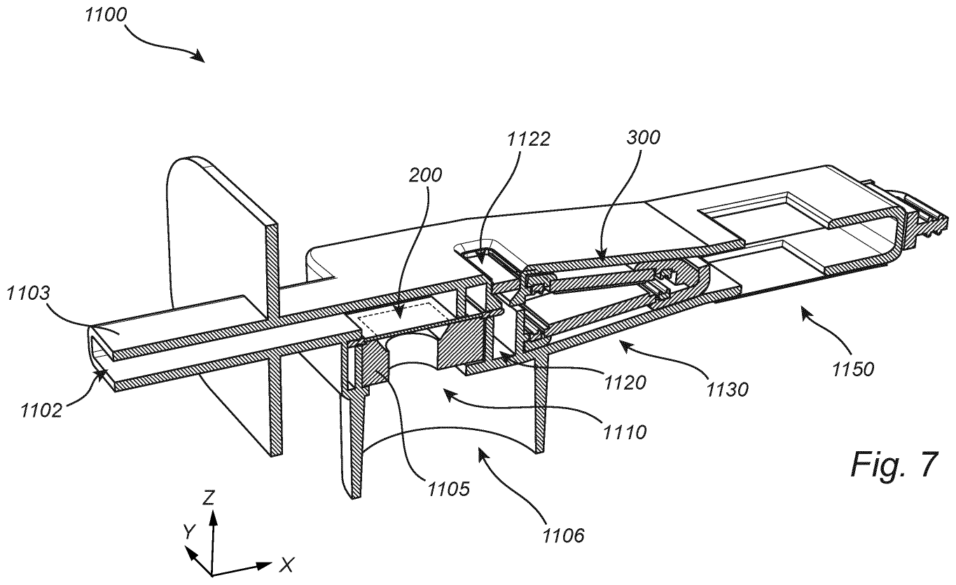

FIG. 7 is a schematic perspective cross-sectional view of a sample collector 1100 according to a further embodiment. The sample collector 1100 is similar to the sample collector 100 and comprises a sampling compartment 1110, a reagent filling compartment 1120 provided with a reagent inlet 1122, an additional compartment 1130 and an analysis compartment 1150. The sample collector 1100 may as shown comprise e.g. the particle capturing substrate 200 and the clamping device 300 as described above. In contrast to the sample collector 100, the sample collector 1100 comprises an inlet 1102 with an integrally formed mouthpiece 1103. Additionally, the inlet 1102 is oriented to receive a flow of air along the length dimension X. Moreover, a thermal body 1105 corresponding thermal body 105 is arranged after the particle capturing substrate 200 as seen from the inlet 1102. The thermal body 110 is hence configured to mainly heat the particle capturing substrate 200, and not also the flow of air prior to reaching the particle capturing substrate 200.

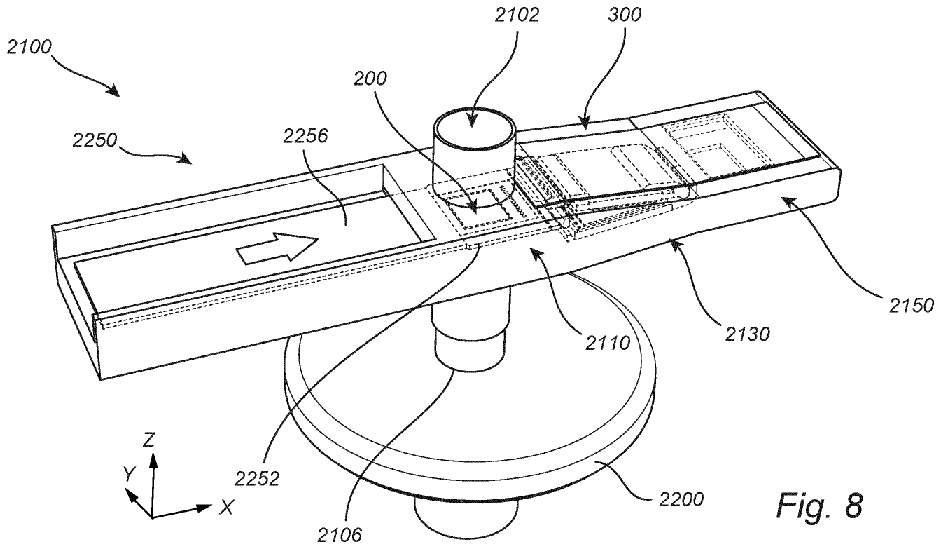

FIG. 8 is a schematic perspective cross-sectional view of a sample collector according to yet a further embodiment. The sample collector 2100 is similar to the sample collector 100 and 1100 and comprises a sampling compartment 2110, an additional compartment 2130 and an analysis compartment 2150. The sample collector 100 may as shown comprise e.g. the particle capturing substrate 200 and the clamping device 300 as described above. In contrast to the sample collector 100, the sample collector 2100 omits a separate reagent filling compartment. Instead, a reagent feed 2122 is provided at the sampling compartment 2110. Additionally, rather than comprising a substrate holder 250 configured to pull the particle capturing substrate 200 from the sampling compartment 2110, the sample collector 2100 comprises a substrate holder 2250 configured to push the particle capturing substrate 200 from the sampling compartment 2110 towards the analysis compartment 2150. Reference sign 2252 denotes a frame portion of the substrate holder 2250. Reference sign 2256 denotes a combined coupling member and gripping part of the substrate holder 2250.

Moreover, FIG. 8 illustrates how auxiliary equipment, such as spirometer 2200, may be connected to the outlet 2106 of the sample collector 2100.

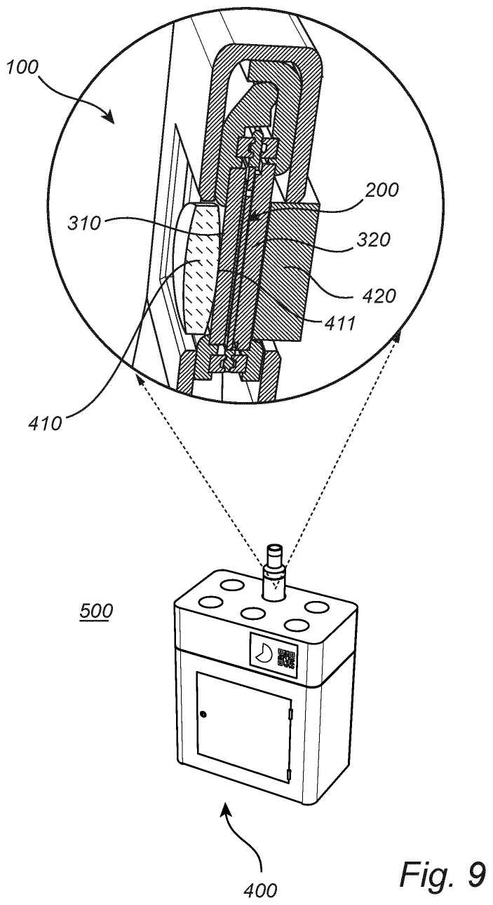

Referring now to FIG. 9, there is shown a system or kit of parts 500 comprising: a sample collector, e.g. sample collector 100; and an analysis instrument 400 configured to receive the sample collector 100 for optical analysis of the airborne particles captured by the particle capturing substrate 200. Although shown to comprise the sample collector 100, the kit of parts 500 may also comprise any of the sample collectors 1100 and 2100.

The analysis instrument 400 comprises optics 410, configured to be aligned with the optical window 310 of the sample collector 100 for optical access to the particle capturing substrate 200 when the sample collector 100 is received in the analysis instrument 400 and the particle capturing substrate 200 is arranged in the analysis position.

The analysis instrument 400 may further comprise a heat source 420, and the support member 320 of the clamping device 300 may be a thermal surface configured to allow thermal energy to be supplied from the heat source 420 of the analysis instrument 400 to the analysis compartment 150 of the sample collector 100, when the sample collector 100 is received in the analysis instrument 400 and the sample collector 100 is in the analysis configuration.

The front surface 411 of the optics 410 of the analysis instrument 400 is configured to abut and press the optical window 310 towards the support member 320, such that the particle capturing substrate 200 is clamped therebetween.

By the analysis instrument 400 being configured to press the optical window 310 towards the support member 320, the particle capturing substrate 200 may be pressed between the support member 320 and the optical window 310 at an increased pressure level compared to the pressure level provided by the clamping device 300 in isolation. The increased pressure level may further enhance sealing of the particle capturing substrate 200, contributing to a reduced risk of contamination. The increased pressure level may further contribute to an efficient analysis with minimized optical artifacts by reducing presence of air gaps between the optical window and the particle capturing substrate.

Thermal energy supplied from the heat source to the analysis compartment 150 allows heat-induced reactions on the particle capturing substrate 200. Heat-induced reactions may facilitate analysis of the collected particles on the particle capturing substrate 200. The collected particles may for instance comprise nucleic acids such as RNA or DNA, which may be exposed by a heat-induced reaction such as thermal lysis. Exposed nucleic acids can further be replicated by a process of cyclic heating and cooling, and thereby amplified to a large amount, facilitating measurements to detect RNA and/or DNA from pathogens, such as an influenza virus or SARS-COV-2.

It should be realized that the particle capturing substrate 200 may be supplied with a reagent, especially a liquid reagent as discussed above. The reagent may be selected from suitable reagents for a certain type of analysis, and may for instance be activated at elevated temperatures.

The measurements may comprise detecting light emitted through luminescence. For instance, the light-based measurement may comprise detecting light being emitted based on stimulation by light, such that the light-based measurement may comprise detecting light emitted through fluorescence. The light-based measurement may for instance be configured to determine a quantity of light, such as a quantity of fluorescent light.

After the particles have been collected, thermal energy may be provided for thermal lysis to expose RNA of severe acute respiratory syndrome coronavirus 2 (SARS-COV-2) in the collected particles. If RNA is converted to DNA using for instance reverse transcriptase based on the reagent, the thermal energy may further facilitate thermal cycling for amplification of the DNA using quantitative polymerase chain reaction. The light-based measurement may then be performed to detect fluorescently tagged DNA formed through the reverse transcriptase quantitative polymerase chain reaction. Although analysis for determining presence of SARS-COV-2 is mainly described, it should be realized that the sample collector 100, analysis instrument 400 and methods may alternatively be used for determining presence of another particle, such as determining presence of influenza particles, or for a completely different type of analysis of captured particles.

In the above the inventive concept has mainly been described with reference to a limited number of examples. However, as is readily appreciated by a person skilled in the art, other examples than the ones disclosed above are equally possible within the scope of the inventive concept, as defined by the appended claims.

Although the embodiments as disclosed with reference to e.g. FIGS. 1-5 comprise a thermal body 105, a thermal body may be omitted from a sample collector, e.g. if condensation on the particle capturing substrate is not a concern, or if heating of the flow of air and/or particle capturing substrate by other means are available.

Additionally, although e.g. the sample collectors 100, 1100, 2100 comprise reagent feeds 122, 1122, 2122, reagent feeds, reagent filling compartments and substrate reagent inlets may be omitted, in case reagent filling is not required in the analysis technique to be used.

It is further envisaged that a sample collector may comprise an additional compartment (like 130) and an analysis compartment (like 150) arranged on opposite sides of a sampling compartment (like 110). Hence, reconfiguration from a sampling configuration to an analysis configuration may comprise moving a clamping device (like 300) into the sampling compartment to receive the particle capturing substrate (like 200) arranged therein, and thereafter further moving the clamping device together with the particle capturing substrate into the analysis compartment while the clamping device is brought to a closed state to clamp the particle capturing substrate. In such a case, a gripping part may be provided which is coupled to the clamping device such that the clamping device may be moved, e.g. pulled, into the sampling compartment.

It is further envisaged that an additional compartment may be omitted. Rather, the clamping device may be arranged in the sampling compartment also in the sampling configuration. The particle capturing substrate may be arranged between the optical window and the support member of the clamping device. The substrate clamping device may be in an open state such that passage of the flow of air through the particle capturing substrate is allowed. The particle capturing substrate may subsequently be moved, together with the clamping device towards the analysis compartment to re-configure the sample collector into the analysis configuration.

1.-24. (canceled)

25. A sample collector for collection of airborne particles exhaled by a human being, comprising:
a sampling compartment comprising an inlet for receiving a flow of air;
a particle capturing substrate configured to capture the airborne particles in the flow of air;
an analysis compartment wherein an aperture is defined in a wall of the analysis compartment; and
a substrate clamping device comprising an optical window and a support member;
wherein the sample collector is reconfigurable between a sampling configuration and an analysis configuration, wherein:
in the sampling configuration the particle capturing substrate is arranged in the sampling compartment such that passage of the flow of air through the particle capturing substrate is allowed; and
in the analysis configuration the substrate clamping device and the particle capturing substrate are arranged in the analysis compartment, the optical window is aligned with the aperture such that optical access to the particle capturing substrate is allowed, and the substrate clamping device is in a closed state such that the particle capturing substrate is clamped between the optical window and the support member; and
wherein, during reconfiguration from the sampling configuration to the analysis configuration, the substrate clamping device and the particle capturing substrate are configured to be moved together to the analysis compartment while the substrate clamping device is brought to the closed state.

26. The sample collector according to claim 25, wherein during reconfiguration from the sampling configuration to the analysis configuration, the substrate clamping device and the particle capturing substrate are configured to be moved together in a sliding movement along first and second guide surfaces of the sample collector, wherein the first and second guide surfaces are configured to engage with the optical window and the support member during the sliding movement to press the optical window and the support member towards each other.

27. The sample collector according to claim 25, wherein the optical window and the support member are pivotably connected to each other.

28. The sample collector according to claim 25, wherein the particle capturing substrate comprises a set of air inlets and set of air outlets, the set of air inlets and the set of air outlets being provided on opposite sides of the particle capturing substrate, wherein the optical window and the support member are configured to cover a respective one of the set of air inlets and the set of air outlets when the particle capturing substrate is in the analysis configuration.

29. The sample collector according to claim 25, further comprising an additional compartment, wherein in the sampling configuration, the substrate clamping device is arranged in the additional compartment.

30. The sample collector according to claim 25, wherein in the sampling configuration, the substrate clamping device is arranged in the sampling compartment and the particle capturing substrate is arranged between the optical window and the support member of the substrate clamping device, the substrate clamping device being in an open state such that passage of the flow of air through the particle capturing substrate is allowed.

31. The sample collector according to claim 25, wherein the substrate clamping device comprises a first sealing structure arranged on and configured to seal the substrate clamping device when the sample collector is in the analysis configuration.

32. The sample collector according to claim 31, wherein the first sealing structure comprises a first sealing member arranged to extend along an entire circumference of the optical window and a second sealing member arranged to extend along an entire circumference of the support member.

33. The sample collector according to claim 32, wherein, when the sample collector is in the analysis configuration, the first sealing member is configured to circumferentially enclose a surface region of a first side of the particle capturing substrate in which a set of air inlets or outlets are provided, and the second sealing member is configured to circumferentially enclose a surface region of a second side of the particle capturing substrate in which a set of air outlets or inlets are provided.

34. The sample collector according to claim 32, wherein the sealing members are configured to define a respective sealed volume between the sealing member and a peripheral edge of the particle capturing substrate, when the sample collector is in the analysis configuration.

35. The sample collector according to claim 25, wherein the particle capturing substrate is an aerosol impactor substrate.

36. The sample collector according to claim 25, further comprising a reagent feed for receiving reagent to be supplied to the particle capturing substrate.

37. The sample collector according to claim 36, wherein the particle capturing substrate is further reconfigurable from the sampling configuration to a reagent filling configuration, wherein the particle capturing substrate is arranged to:
be aligned with the reagent feed; and
receive the reagent through the reagent feed.

38. A kit of parts comprising:
a sample collector according to claim 25; and
an analysis instrument configured to receive the sample collector for optical analysis of the airborne particles captured by the particle capturing substrate,
wherein, when the sample collector is received in the analysis instrument and the particle capturing substrate is arranged in the analysis configuration,
optics of the analysis instrument is configured to be aligned with the optical window of the sample collector for optical access to the particle capturing substrate while the analysis instrument is configured to press the optical window towards the support member such that the particle capturing substrate is clamped therebetween.

39. A method for collection of airborne particles exhaled by a human being with a sample collector according to claim 25, the method comprising:
receiving via the inlet of the sample collector a flow of air, the flow of air carrying the airborne particles;
capturing the airborne particles in the flow of air from the inlet by the particle capturing substrate while the sample collector is configured in the sampling configuration; and
reconfiguring the sample collector to the analysis configuration wherein the substrate clamping device and the particle capturing substrate are arranged in the analysis compartment, the optical window is aligned with the aperture such that optical access to the particle capturing substrate is allowed, and the substrate clamping device is in the closed state such that the particle capturing substrate is clamped between the optical window and the support member;
wherein the reconfiguring step comprises moving the substrate clamping device and the particle capturing substrate together to the analysis compartment while the substrate clamping device is brought to the closed state.

40. The method according to claim 39, wherein the reconfiguring step comprises moving the substrate clamping device and the particle capturing substrate together in a sliding movement along first and second guide surfaces of the sample collector configured to engage with the optical window and the support member during the sliding movement to press the optical window and the support member towards each other.

41. The method according to claim 39, wherein the sample collector further comprises an additional compartment, wherein in the sampling configuration, the substrate clamping device is arranged in the additional compartment. wherein the additional compartment is arranged intermediate the sampling compartment and the analysis compartment, and wherein, during reconfiguration from the sampling configuration to the intermediate configuration, the particle capturing substrate is configured to be moved to the additional compartment to be received between the optical window and the support member, and thereafter be moved, together with the substrate clamping device, to the analysis compartment, wherein the reconfiguring step comprises moving the particle capturing substrate from the sampling compartment to the additional compartment to be received between the optical window and the support member, and thereafter moving the substrate clamping device and the particle capturing substrate together to the analysis compartment.

42. The method according to claim 39, wherein in the sampling configuration, the substrate clamping device is arranged in the sampling compartment and the particle capturing substrate is arranged between the optical window and the support member of the substrate clamping device, the substrate clamping device being in an open state such that passage of the flow of air through the particle capturing substrate is allowed, wherein the reconfiguring step comprises moving the substrate clamping device and the particle capturing substrate together from the sampling compartment to the analysis compartment.

43. The method according to claim 39, wherein the sample collector further comprises a reagent feed for receiving reagent to be supplied to the particle capturing substrate, the method further comprising supplying a reagent to the particle capturing substrate by introducing a reagent through the reagent feed of the sample collector.

44. The method according to claim 43, wherein the particle capturing substrate is further reconfigurable from the sampling configuration to a reagent filling configuration, wherein the particle capturing substrate is arranged to: be aligned with the reagent feed; and receive the reagent through the reagent feed, the method further comprising, subsequent to the step of capturing, reconfiguring the sample collector from the sampling configuration to the reagent filling configuration, and subsequent to the step of supplying the reagent, reconfiguring the sample collector to the analysis configuration.
